(19) United States
(12) Patent Application Publication
(10) Pub. No.: US 2019/0380855 A1
(43) Pub. Date: Dec. 19, 2019

(54) APPETITE CONTROL DEVICE

(71) Applicant: Kendall Ann Whitney, Westlake Village, CA (US)

(72) Inventor: Kendall Ann Whitney, Westlake Village, CA (US)

(21) Appl. No.: 16/341,670

(22) PCT Filed: Oct. 12, 2017
(86) PCT No.: PCT/US17/56381

§ 371 Date: Apr. 12, 2019

Related U.S. Application Data
(60) Provisional application No. 62/407,128, filed on Oct. 12, 2016.

(51) Int. Cl. A61F 5/00; A24B 15/24; A23L 33/10

(52) U.S. Cl. CPC A61F 5/0006; A23V 2002/00; A23L 33/10; A24B 15/243

(57) ABSTRACT

An oral appliance comprising an appetite control device for covering a plurality of teeth, wherein the appetite control device comprises an overall arcuate shape having an outer sidewall, an inner sidewall, and a top wall connecting the inner sidewall and outer sidewall to form a channel, wherein the channel is configured for receiving a plurality of teeth, wherein the appetite control device comprises a larger width at ends and a narrower width at a front of the appetite control device.

### CROSS-REFERENCE TO RELATED APPLICATIONS

This application is a 35 U.S.C. § 371 National Stage Entry of International Application No. PCT/US2017/056381, filed Oct. 12, 2017, which claims the priority benefit of U.S. Provisional Patent Application Ser. No. 62/407,128, filed Oct. 12, 2016, all of which are incorporated herein by reference in their entirety for all purposes.

### TECHNICAL FIELD

The invention, in its several embodiments, pertains to health aides, and more particularly to a dieting device.

### BACKGROUND

Existing ways to lose weight include dieting, diet pills, exercise, and weight loss surgery. Many people start and stop diets to attempt to lose weight and/or curb overeating with minimal success. Some individuals turn to weight loss surgery, such as gastric bypass, which is dangerous and is not guaranteed to result in a loss of weight and/or change of habits.

### SUMMARY

An exemplary apparatus comprises an oral appliance including an appetite control device for covering a plurality of teeth, wherein the appetite control device comprises an overall arcuate shape having an outer sidewall, an inner sidewall, and top wall connecting the inner sidewall and outer sidewall, together forming a channel, where the channel is configured for receiving a plurality of teeth. In one embodiment, the appetite control device comprises a larger width at ends and a narrower width at a front of the appetite control device. The apparatus may further comprise: a flavor pouch disposed on the appetite control device between a left end and a right end of the appetite control device; where the flavor pouch is sized to hold one or more flavor tabs or flavor gels to deliver one or more flavors to a user via contact with a roof of a user's mouth

The exemplary apparatus may also include a lower appetite control device for covering a plurality of lower teeth. The lower appetite control device may be dimensioned to cover fourteen lower teeth of the user. The lower appetite control device may be dimensioned to cover between eight and twelve lower teeth of the user. The appetite control device may be dimensioned to cover fourteen upper teeth of the user. The appetite control device may be dimensioned to cover between eight and twelve upper teeth of the user. The apparatus may include a storage case, where the storage case may be dimensioned to hold the appetite control device, the lower appetite control device, and one or more flavor tabs or flavor gels. The appetite control device may be made from a translucent plastic.

The appetite control device may comprise one or more apertures on an outer sidewall and/or one or more apertures on an inner sidewall. The appetite control device may have a generally trapezoidal cross section. The flavor tab or flavor gel may include nicotine. A top surface of the flavor pouch may be substantially level with an upper surface of the appetite control device. A lower surface of the flavor pouch may be substantially level with a lower surface of the appetite control device. The flavor pouch may be disposed between an upper surface and a lower surface of the appetite control device. The appetite control device may be made from a heat-cured acrylic resin. The appetite control device may be made from a heat-contoured laminate material. The appetite control device may be made from a dual-laminate material comprising a hard acrylic outside and a soft laminate inside.

The appetite control device may comprise one or more retention mechanisms to increase retention forces on a user's teeth. The flavor pouch may include a plurality of apertures. The plurality of apertures may be disposed on at least one of: a top surface of the flavor pouch and a bottom surface of the flavor pouch. A size of the plurality of apertures may be dimensioned based on a desired longevity of the flavor tabs or flavor gels in the flavor pouch. The flavor pouch may be detachably attached to the appetite control device. The appetite control device may comprise one or more detachable attachment fasteners for receiving the flavor pouch. The flavor pouch may comprise one or more fasteners for receiving the one or more detachable attachment fasteners of the appetite control device. The one or more fasteners may comprise at least one of: a protrusion, a hook, and an indentation. The flavor pouch may comprise surface indicia. The surface indicia may be disposed on a top surface of the flavor pouch. The surface indicia may be disposed on a bottom surface of the flavor pouch.

Another exemplary device embodiment may comprise: a lower appetite control device for covering a plurality of lower teeth; an upper appetite control device for covering a plurality of upper teeth; and a flavor pouch sized to hold one or more flavor tabs to deliver one or more flavors to a user via contact with a roof of a user's mouth; where the flavor pouch may be detachably attached to the upper appetite control device between a left end and a right end of the upper appetite control device by one or more fasteners of the flavor pouch and one or more attachments of the upper appetite control device. A top surface of the flavor pouch may be substantially level with an upper surface of the upper appetite control device. A lower surface of the flavor pouch may be substantially level with a lower surface of the upper appetite control device. The flavor pouch may be disposed between an upper surface and a lower surface of the upper appetite control device. The flavor pouch may comprise a plurality of apertures.

An exemplary system embodiment may comprise: a flavor pouch sized to hold one or more flavor tabs to deliver one or more flavors to a user via contact with a roof of a user's mouth; where the flavor pouch comprises surface indicia disposed on a lower surface of the flavor pouch. The system may further comprise an upper appetite control device for covering a plurality of upper teeth; where the flavor pouch is detachably attached to the upper appetite control device between a left end and a right end of the upper appetite control device by one or more fasteners of the flavor pouch and one or more attachments of the upper appetite control device. The system may further comprise an upper appetite control device for covering a plurality of upper teeth; where the flavor pouch may be fixedly attached to the upper appetite control device between a left end and a right end of the upper appetite control device.

### BRIEF DESCRIPTION OF THE DRAWINGS

Embodiments of the present invention are illustrated by way of example and not limitation in the figures of the accompanying drawings, which may not be drawn to scale, and in which:

FIG. 1 shows a top view of an example oral appliance comprising an appetite control device for covering teeth, according to one embodiment;

FIG. 2 shows another example appetite control device having a flavor pouch, according to one embodiment;

FIG. 3 shows a perspective view of the appetite control device in FIG. 1, according to one embodiment;

FIG. 4 shows a cross-sectional view of the appetite control device in FIG. 1 about line G-G, according to one embodiment;

FIG. 5 shows a bottom view of the appetite control device in FIG. 1, according to one embodiment;

FIG. 6 shows a top view of an appetite control device having a flavor pouch, according to one embodiment;

FIG. 7 shows a perspective view of an appetite control device having a flavor pouch, according to one embodiment;

FIG. 8A shows a perspective view of an appetite control device having a flavor pouch and covering a minimal number of teeth, according to one embodiment;

FIG. 8B shows a perspective view of an appetite control device covering a minimal number of teeth, according to one embodiment;

FIG. 8C shows a perspective view of an appetite control device only covering a lower portion of a user's front teeth, according to one embodiment;

FIG. 8D shows a perspective view of an appetite control device only covering a lower portion of a user's back teeth, according to one embodiment;

FIG. 8E shows a view of an appetite control device only covering a lower portion of a user's front teeth, according to one embodiment;

FIG. 8F shows a view of an appetite control device covering a user's front teeth, according to one embodiment;

FIG. 8G shows a perspective view of an appetite control device covering a user's back teeth, according to one embodiment;

FIG. 9 shows a perspective view of an appetite control device having a flavor pouch with a plurality of apertures, according to one embodiment;

FIG. 10 shows a perspective view of an appetite control device having a flavor pouch made from a mesh, according to one embodiment;

FIG. 11 shows a perspective view of an appetite control device having a detachable flavor pouch with the detachable flavor pouch detached, according to one embodiment;

FIG. 12 shows a perspective view of the appetite control device of FIG. 11 having the detachable flavor pouch attached, according to one embodiment;

FIG. 13 shows a perspective view of an appetite control device having a flavor pouch with surface indicia, according to one embodiment; and

FIG. 14 shows a perspective view of an example appetite control device having a plurality of apertures, according to one embodiment.

### DETAILED DESCRIPTION

The description herein is made for the purpose of illustrating the general principles of the embodiments disclosed herein and is not meant to limit the concepts disclosed herein. Further, particular features described herein can be used in combination with other described features in each of the various possible combinations and permutations. Unless otherwise specifically defined herein, all terms are to be given their broadest possible interpretation including meanings implied from the description as well as meanings understood by those skilled in the art and/or as defined in dictionaries, treatises, etc.

Embodiments of an oral appliance comprising an appetite control device is disclosed herein wherein the appetite control device may be worn by placing it in the mouth on all, or part, of the upper and/or lower teeth by a user. The appetite control device is a physical impediment to eating and/or snacking between meals as it must be removed prior to eating. The appetite control device also makes the user think about not eating by making the user more aware of their behavior. In order to eat food, or snack between meals, the user must consciously remove the upper and/or lower appetite control devices from their teeth prior to snacking. The user then consciously places the upper and/or lower appetite control devices back in their mouth after eating and/or snacking. In some embodiments, the appetite control device may contain a flavor pouch. The flavor pouch may receive a flavored tab such as mint, lemon, cherry, or bubblegum. The appetite control device may be stored in a compact case when not in use.

The appetite control device may be used in conjunction with diet and exercise, without negative consequences or side effects. While other weight loss techniques may focus on fewer calories in foods, diet pills, and/or weight loss surgery, these techniques do not address the behavioral component in weight loss, which is addressed by the use and design of the embodiments of the oral appliance comprising an appetite control device disclosed herein. The appetite control device is a behavioral modification tool that makes users more conscious of what they put in their mouths. Users may decide when to use the appetite control device, such as for appetite control, smoking cessation, etc. In one embodiment, the appetite control device is small and discrete, and so does not alert others that the user is wearing the appetite control device and/or dieting. Users can continue to speak and drink without impediment while wearing the appetite control device in the mouths.

In one embodiment, an appetite control device disclosed herein comprises an overall arcuate shape (e.g., U-shaped) having an outer sidewall 112, an inner sidewall 114, and a top wall 105 connecting the inner sidewall 114 and outer sidewall 112 to form a channel 510, wherein the channel is configured for receiving at least a portion of a plurality of teeth, wherein the appetite control device comprises a larger width at ends and a narrower width at a front of the appetite control device.

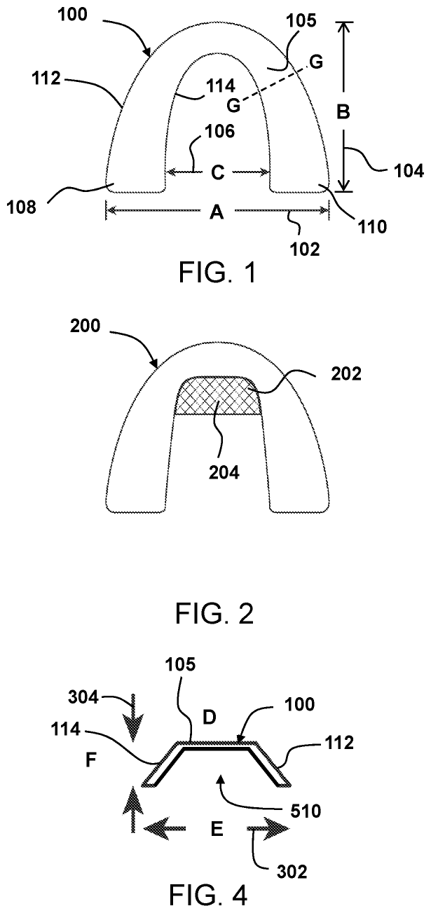

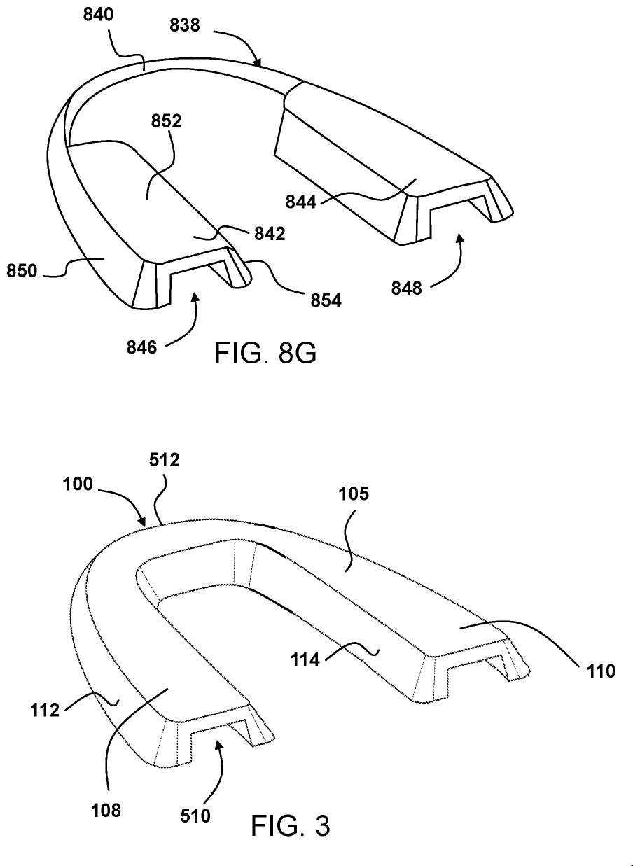

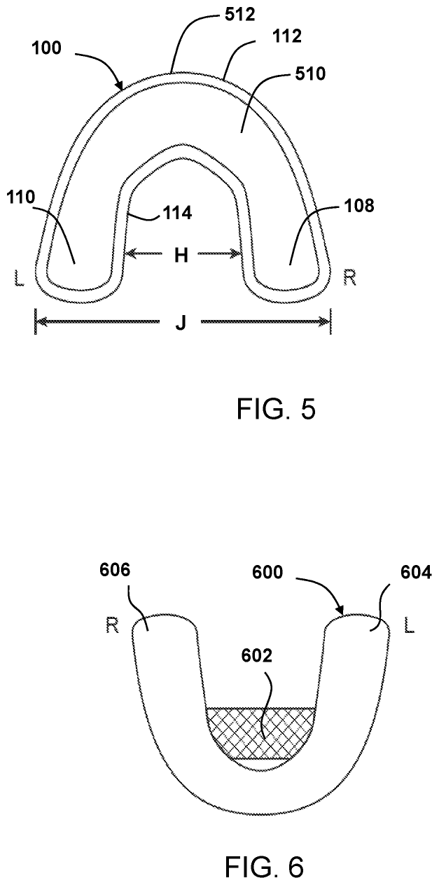

FIG. 1 shows a top view of an oral appliance/apparatus 100 comprising a generally arcuate appetite control device, according to one embodiment. FIG. 5 shows a bottom view of the appetite control device 100 in FIG. 1. FIG. 3 shows a perspective view of the appetite control device 100 of FIG. 1, and FIG. 4 shows a cross-section of the appetite control device 100 of FIG. 1, along line G-G. Referring to the embodiments shown in FIGS. 1 and 3-5 the appetite control device 100 comprises an essentially U-shaped oral appliance having walls defining a trough or channel 510 (e.g., FIG. 5) for covering at least a portion of human teeth, such as a portion of the lower teeth. The channel has an inner sidewall 114, an outer sidewall 112, and a top wall 105 connecting the sidewalls 112, 114. The top wall 105 is transverse to the sidewalls 112, 114, and the sidewalls 112, 114 may be generally transverse in relation to each other where they meet the top wall 105.

When the appetite control device 100 is placed in the mouth as worn by a user, the channel 510 top wall 105 covers the top of surface of the teeth, the channel outer sidewall 112 is positioned between the inner surface of the cheek/lip tissue and the outer surface of the teeth, and the channel inner sidewall 114 is positioned between the periphery of the tongue and the inner surface of the teeth.

Though the appetite control device 100 is described in this embodiment as being useful with lower teeth, the device 100 can also be used on upper teeth. Further, two appetite control devices 100 can be used at the same time, one on the upper teeth and one on the lower teeth. A lower appetite control device may be identical to an upper appetite control device that is worn on the upper teeth, and as such embodiments of the appetite control device are described herein by way of example. In one embodiment, the walls 112, 114 of the appetite control device 100 may be sized and shaped such that the channel 510 of the appetite control device may cover all of the teeth, or cover only a portion (a set) of the teeth such as only the front teeth or side teeth, as described in more detail hereinbelow.

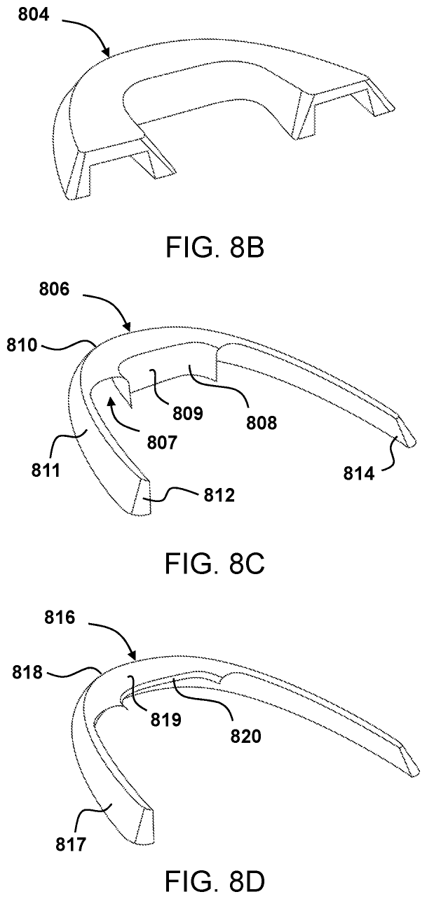

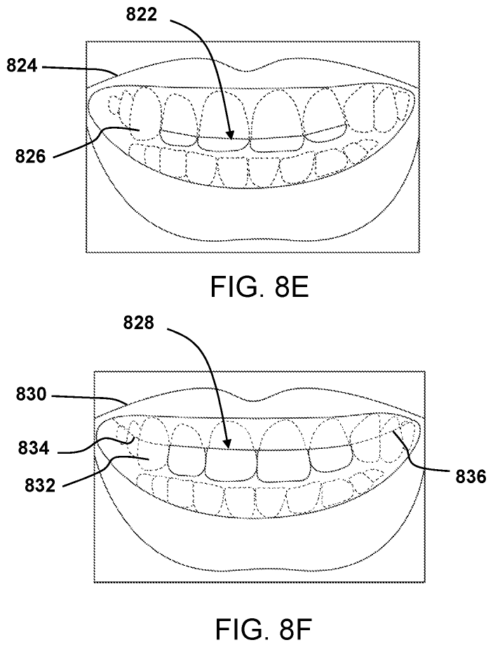

In other embodiments of the appetite control device 100 disclosed herein, the channel 510 is configured to cover a portion of the lower teeth (or upper teeth), such as: only the central incisor (front) set of teeth, or only the lateral incisor set of teeth, or only the canine (cuspid) set teeth, or only the premolar (bicuspid) set of teeth, or only the molar set of teeth. In other embodiments, the channel 510 is configured to cover a combination of one or more of the above set of teeth. For example, in one embodiment the channel 510 may be configured to cover only the premolar and molar sets of teeth, and not cover the central incisor and lateral incisor sets of teeth (FIG. 8G). In another embodiment the channel 510 may be configured to cover only the central incisor and lateral incisor sets of teeth, and not cover the premolar and molar sets of teeth (FIG. 8B). Yet, in another embodiment, the channel 510 may be configured to cover only the central incisor set of teeth, but not cover the lateral incisor, the premolar and molar sets of teeth.

In one embodiment, the appetite control device 100 may be made from a soft and flexible, or firm and hard, clear or colored plastic. In one embodiment, the appetite control device 100 may be flavored or non-flavored. In one embodiment, the appetite control device 100 may be a non-toxic, reusable, and user removable oral appliance worn by a user to prevent overeating and/or snacking between meals. In one embodiment, the appetite control device 100 may be disposable.

In one embodiment, the appetite control device 100 may be customized to fit each user snuggly and comfortably, such as by a heating process to mold a portion of the appetite control device 100 to the user's teeth. The appetite control device 100 may be heated in a microwave oven, heated water, etc. The molded appetite control device 100 may be a smooth firm plastic that fits snugly against the user's teeth once molded. The appetite control device 100 may be stored in a compact plastic case when not in use. This plastic case may be easily stored in the user's purse or pocket. The appetite control device 100 may be made from a translucent plastic so as to be discreet.

In one embodiment shown in FIG. 1, the appetite control device 100 may have an outer width A 102 of about 5.715 cm, a height B 104 of about 5.08 cm, and an inner width C 106 between left end 110 and right end 108 of about 3.175 cm. In some embodiments, the channel 510 of the appetite control device 100 may be wider at the left end 110 and right end 108 by the location of the back molars of the user. In other embodiments, the appetite control device 100 may contain one or more apertures 1402 (FIG. 14) on an outer sidewall 112, one or more apertures 1404 (FIG. 14) on an inner sidewall 114, and/or one or more apertures 1406 (FIG. 14) on a top wall 105 for ventilation, saliva flow, cleaning, and/or drying. An upper and/or lower appetite control device may contain one or more apertures on an outer sidewall 112 and/or inner sidewall 114.

FIG. 2 shows an embodiment of the disclosed oral appliance comprising an appetite control device 200 having a flavor pouch 202 for covering all or a portion of the upper teeth. The device 200 can similarly be configured for use on lower teeth. The appetite control device 200 may be identical to the appetite control device 100 of FIG. 1, except for the addition of a flavor pouch 202. The flavor pouch 202 may be used to hold one or more flavor tabs 204 and/or flavor gels. The flavor tabs 204 may be inserted into the flavor pouch 202 and provide one or more flavors, such as mint, cherry, or bubblegum, to a user via the roof of their mouth.

FIG. 3 shows a perspective view of the appetite control device 100 in FIG. 1. The channel 510 of the appetite control device 100 may be formed by the outer sidewall 112, inner sidewall 114, and top wall 105. The appetite control device includes a front 512 and ends 108, 110.

FIG. 4 shows a cross-sectional view of the appetite control device 100 in FIG. 1 about line G-G. The appetite control device 100 may have a generally trapezoidal cross section, such as an isosceles trapezoid, forming said channel 510. In one example, the appetite control device 100 may have a lower width D of about 0.635 cm, an upper width E 302 of about 1.27 cm, and a height F 304 of about 0.47625 cm. In some embodiments, the appetite control device 100 may be thinner. Other example dimensions are possible. The dimensions of the appetite control device can be configured based on the oral characteristics, mouth size, teeth size and position, etc., to comfortably fit in the mouth of a user.

FIG. 5 shows a bottom view of the appetite control device 100 having a left side 110 and a right side 108. The appetite control device 100 may have an outer sidewall 112 and an inner sidewall 114. In some embodiments, the sidewalls 112, 114 may surround a perimeter of the appetite control device 100. The inner width H may be 3.175 cm and the outer width J may be 5.715 cm. The upper and/or lower appetite control device may have an overall U-shape with the outer sidewall 112 and inner sidewall 114 surrounding the channel 510 for receiving teeth of a user. The upper and/or lower appetite control device may have a larger width at ends 108, 110 and a narrow width at front 512. The thickness of the sidewalls 112, 114 and top wall 105 forming the channel 510 may be minimal (e.g., between 0.25 mm to 3 mm, and preferably between 0.5 mm to 1 mm) compared to a conventional mouth guard, sufficient to maintain the appetite control device in the user's mouth while engaging a portion of the teeth, and inhibit eating or drinking rather than to protect the teeth from grinding, impact, etc. Other example dimensions are possible. The dimensions of the appetite control device can be configured based on the oral characteristics, mouth size, teeth size and position, etc., to comfortably in the mouth of a user.

FIG. 6 shows another embodiment of the oral appliance comprising an appetite control device 600 having a flavor pouch 602. The appetite control device 600 may have a left side 604 and a right side 606. In some embodiments, the appetite control device may be used as an upper and/or lower appetite control device, and may be used as an anti-smoking device and/or a deterrent to smoking. The flavor pouch 602 may be used to hold one or more nicotine tabs and/or nicotine gels that deliver nicotine to the user.

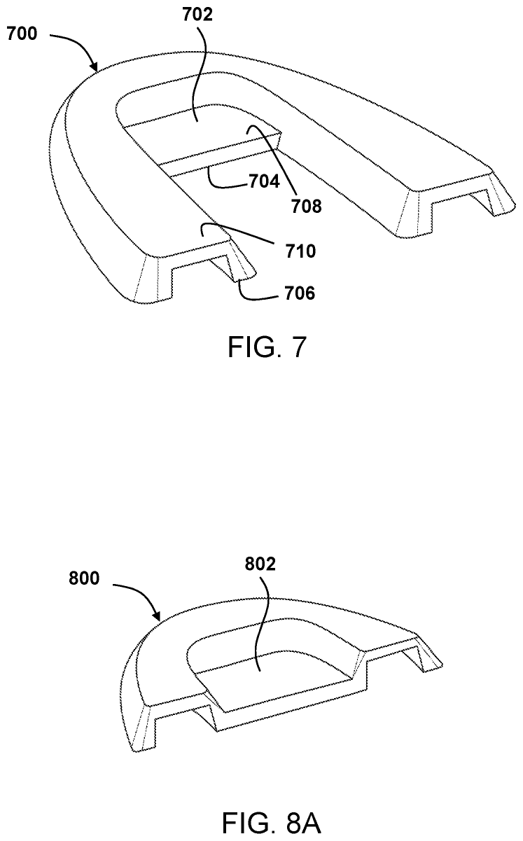

FIG. 7 shows a perspective view of an example upper (or lower) appetite control device 700 having a flavor pouch 702. The appetite control device 700 may be identical to the appetite control device 100, with the addition of the pouch 702. The flavor pouch 702 is shown as having a top surface 704 substantially level with an upper surface 706 of the upper appetite control device 700 so as to be in contact with the roof of a wearer's mouth. In some embodiments, the lower surface 708 of the flavor pouch 702 may be substantially level with the lower surface 710 of the upper appetite control device 700. In other embodiments, the flavor pouch 702 may be disposed between the upper surface 706 and lower surface 710 of the upper appetite control device 700.

FIG. 8A shows a perspective view of an upper (or lower) appetite control device 800 having a flavor pouch 802 and covering a minimal number of teeth. The appetite control device 800 may only extend to cover the front upper teeth of a wearer needed to support the flavor pouch 802 disposed between the ends of the upper appetite control device 800.

In some embodiments, the upper appetite control device 800 may be constructed from a transparent material, e.g., a clear plastic, so as to minimize the visual appearance of the upper appetite control device 800. The upper appetite control device 800 of the appetite control device may be minimal in construction so as to make it difficult to eat or drink rather than to provide an appetite control device or protection to a user's teeth.

FIG. 8B shows a perspective view of an upper (or lower) appetite control device 804, without a flavor pouch, covering a minimal number of teeth. The upper appetite control device 804 may be used to cover only three or four teeth and act as an impediment to eating or drinking rather than as a guard to teeth, e.g., to prevent grinding or protect from impact. When placed in the mouth of a user, compared to the device 700 in FIG. 7, the device 800 in FIG. 8A does not extend to the molar teeth, and only covers the front and canine teeth, for example.

FIG. 8C shows a perspective view of an embodiment of the oral appliance, comprising appetite control device 806 which only covers a portion of a user's front teeth. In use, the front portion 810 of the appetite control device 806 may only cover a portion of the user's front teeth, as depicted in FIG. 8E. The channel 807 defined by the walls 809, 811 of the appetite control device 806 only cover the front teeth of the user, such that the device 806 is supported the mouth over the user's front teeth via a back portion 808 of the inner sidewall 809. The outer sidewall 811 of the appetite control device 806 may extend to the back of a user's mouth along the gum line next to molars via ends 812, 814, to provide more stability in the user's mouth. Ends 812, 814 may be constructed from plastic, metal, a wire, etc. and may prevent the appetite control device from being swallowed, keep the device in place in the mouth, etc. In some embodiments, ends 812, 814 may not be present.

FIG. 8D shows a perspective view of an appetite control device 816 only covering a lower portion of a user's back teeth via back portion 820. The appetite control device 816 may include a front portion 818 of the outer sidewall 817 that covers at least a part of the front of a user's teeth and a portion 820 of the top wall 819 that covers only a portion of the top of a user's teeth, such that the tongue may freely touch the back of the front teeth inside the mouth for easier speech making. In one embodiment, the portion 820 may have a hook, or J, shape to secure against the teeth of a user without inhibiting the user's ability to use their tongue and talk normally. Accordingly, a user may be able to wear the appetite control device 816 without altering others that they are using it as their speech will not be inhibited. However, the user's ability to eat and drink normally will be impacted by using the appetite control device 816.

FIG. 8E shows a view of an upper appetite control device 822 only covering a lower portion of a user's front teeth 826. The upper appetite control device 822 may be inserted into a mouth 824 of a user and only cover a lower portion of the users front teeth 826, such that the visual appearance of the appetite control device is minimized.

FIG. 8F shows a view of an upper appetite control device covering a user's front teeth. The upper appetite control device 828 may be inserted into a mouth 830 of a user and cover at least a portion of front of the user's teeth 832. The back of the user's teeth may only be minimally covered in a hook, or J, shape so as to not inhibit the user's tongue so that the user may pronounce words normally. The appetite control device may include one or more ends 834, 836 shown in dashed lines that may extend to the back of a user's mouth and/or cover additional teeth 832. The ends 834, 836 may be plastic, metal, a wire, etc. and may be used to hold the upper appetite control device 828 in place and/or prevent swallowing the upper appetite control device 828. The upper appetite control device 828 may be used in conjunction with a lower appetite control device, such as shown in FIG. 1. In some embodiments, only a lower appetite control device may be used to minimize visual appearance to others, while still inhibiting drinking and/or eating. The upper appetite control device and lower appetite control device may be identical in some embodiments.

FIG. 8G shows a view of an upper appetite control device 838 covering a user's back teeth. The appetite control device 838 may include an outer wall 850, an inner wall 854, and a top wall 852 forming two channel segments 846, 848, connected by a portion 840 of the outer wall 850. The outer wall 850 may be spaced between an outside surface of a user's molars and a cheek of a user. The inner wall 854 may be spaced between an inner surface of a user's molars and an inside of a mouth of a user. The top wall 852 may be spaced between the top surfaces of the user's molars. The channel segments 846, 848 may surround portions of the user's molars. The front portion 840 of the outside channel 850 may extend to connect the ends 842, 844 of the appetite control device 838. The front portion 840 may be a thin segment which may be disposed between the gums and inner lip of a user where it may not be readily visible to an outside observer. In other embodiments, the front portion 840 may extend to cover a portion of the user's front teeth. The appetite control device may be formed to cover a portion of any of a user's teeth with varying degrees of visibility to an outside observer based on a desired ease of wearing, ease of speaking, ability to prevent eating and/or drinking, etc.

In one embodiment, the appetite control device 100 comprises moldable material that is ergonomically formed for alignment with the bite of the user. The appetite control device 100 may comprise essentially rigid material such as heat-cured acrylic resin, a hard, clear plastic material. The appetite control device 100 may also comprise essentially soft material such as heat-contoured laminate material. In one embodiment, the appetite control device 100 comprises dual-laminate material, such as hard acrylic on the outside and soft laminate material on the inside.

In another embodiment, the appetite control device 100 comprises a thermo-plastic material adapted to fit a user's teeth and gums by heating and molding such as boiling then placing in the mouth. The appetite control device is placed in hot water to soften, then placed in the mouth and shaped around the teeth using finger and tongue pressure. Retention mechanisms such as fins may be included in designated areas which allow increasing retention force on the teeth. In an additional embodiment, the appetite control device 100 comprises Ethylene-vinyl acetate. An impression of the user's teeth may be used to create a properly fitting appetite control device 100. The impression may be obtained using an impression and dental putty.

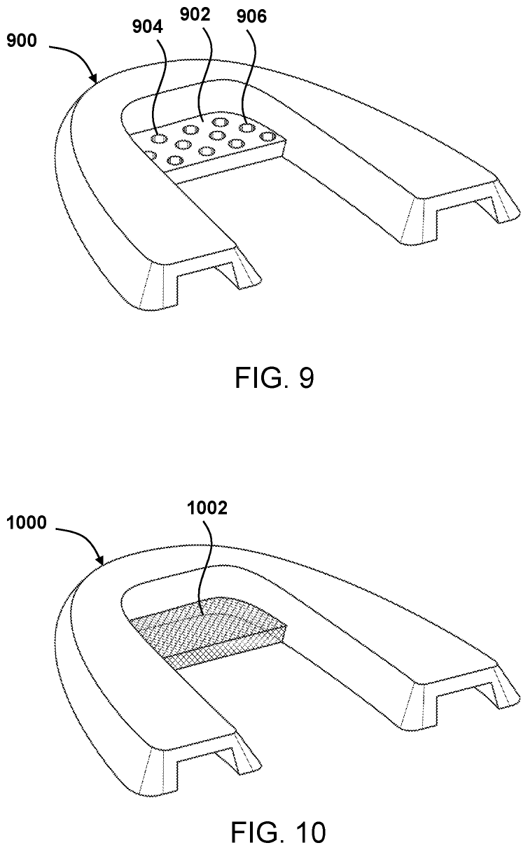

FIG. 9 shows a perspective view of an upper (or lower) appetite control device 900 having a flavor pouch 902 with a plurality of apertures 904, 906. The flavor pouch 902 may have apertures 904, 906 on a top surface, a bottom surface, and/or a side. In some embodiments, the aperture sizes may vary based on the desired longevity of a tab and/or gel placed in the flavor pouch 902.

FIG. 10 shows a perspective view of an upper (or lower) appetite control device 1000 having a flavor pouch made from a mesh 1002. The mesh 1002 may be used to hold a tab and/or flavored gel in a desired position in the user's mouth relative to the upper appetite control device 1000 while allowing the tab to dissolve through the mesh and provide flavor to the user.

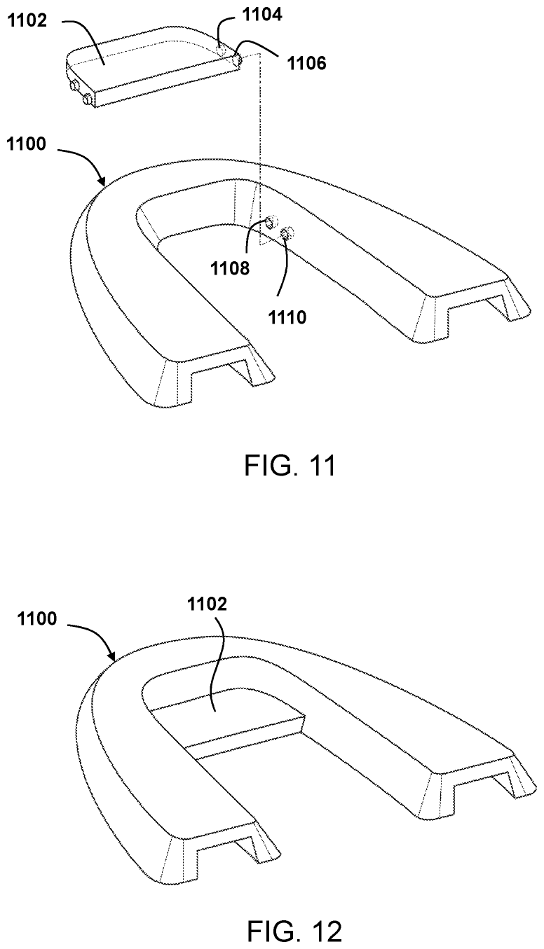

FIG. 11 shows a perspective view of an upper (or lower) appetite control device 1100 having a detachable flavor pouch 1102 with the detachable flavor pouch 1102 detached. FIG. 12 shows a perspective view of the upper (or lower) appetite control device 1100 of FIG. 11 having the detachable flavor pouch 1102 attached. The upper appetite control device 1100 may have one or more detachable attachment fasteners 1108, 1110, such as indentations, hooks, etc. The detachable flavor pouch 1102 may have one or more corresponding fasteners 1104, 1106, such as protrusions, hooks, indentations, etc. for receiving the one or more fasteners 1108, 1110 on the upper appetite control device 1100.

The detachable flavor pouch 1102 may be reusable and a tab and/or gel may be inserted into the flavor pouch 1102. In other embodiments, the detachable flavor pouch 1102 may be disposable and/or dissolve during use. A user may utilize a disposable detachable flavor pouch 1102 and then replace with another disposable detachable flavor pouch 1102 once the first pouch has dissolved. The detachable flavor pouch 1102 may include any of the designs, elements, and/or functions as shown in FIGS. 7-10 and 13.

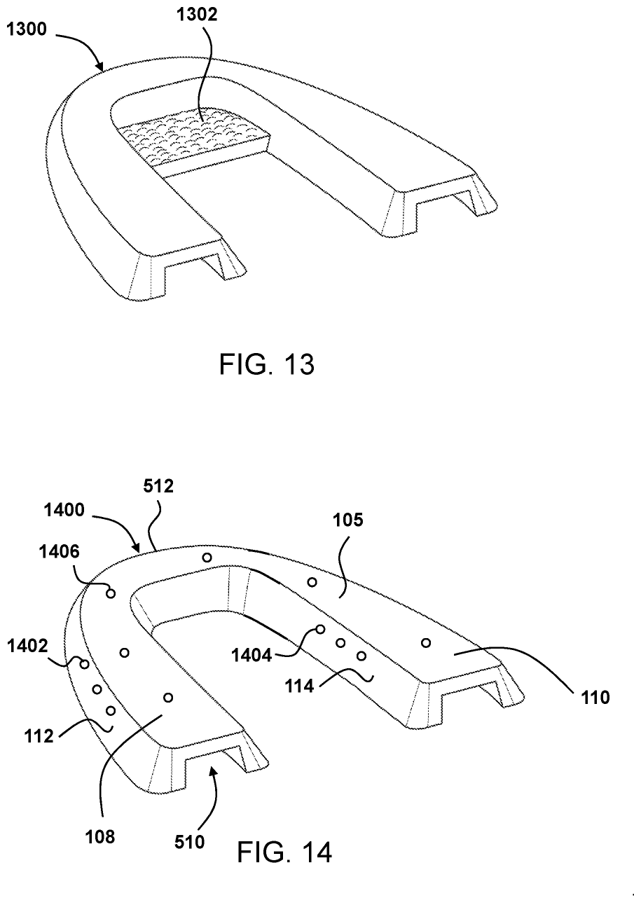

FIG. 13 shows a perspective view of an upper appetite control device 1300 having a flavor pouch with surface indicia 1302. The flavor pouch may have indicia 1302 such as a surface texture. The texture 1302 may be present on a top surface, a bottom surface, and/or side surfaces of the flavor pouch. The texture 1302 may provide feedback to a user, via a tongue and/or roof of a mouth, when the flavor pouch, a tab enclosed within the flavor pouch, and/or a gel enclosed within the flavor pouch needs to be replaced. The texture 1302 may also make the user more aware of the presence of the upper appetite control device 1300 and act to decrease their likelihood of overeating, smoking, etc.

FIG. 14 shows a perspective view of another appetite control device 1400 having a plurality of apertures. The appetite control device 1400 may contain one or more apertures 1402 on an outer sidewall 112, one or more apertures 1404 on an inner sidewall 114, and/or one or more apertures 1406 on a top wall 105 for ventilation, saliva flow, cleaning, and/or drying. An upper and/or lower appetite control device may contain one or more apertures on an outer sidewall 112 and/or inner sidewall 114.

Those skilled in the art will appreciate that various adaptations and modifications of the described preferred embodiments can be configured without departing from the scope and spirit of the improved pressure switch system described herein. Therefore, it is to be understood that, within the scope of the embodiments, the switch system may be practiced other than as specifically described herein.

1. An apparatus comprising:
an appetite control device for covering a plurality of teeth, wherein the appetite control device comprises an overall arcuate shape having an outer sidewall, an inner sidewall and a top wall connecting the inner sidewall and outer sidewall to form a channel, wherein the channel is configured for receiving a plurality of teeth, wherein the appetite control device comprises a larger width at ends and a narrower width at a front of the appetite control device.

2. The apparatus of claim 1, further comprising:
a flavor pouch disposed on the appetite control device between a left end and a right end of the appetite control device;
wherein the flavor pouch is sized to hold one or more flavor tabs or flavor gels to deliver one or more flavors to a user via contact with a roof of a user's mouth.

3. The apparatus of claim 2, further comprising:
a lower appetite control device for covering a plurality of lower teeth.

4. The apparatus of claim 3, wherein the lower appetite control device is dimensioned to cover fourteen lower teeth of the user.

5. The apparatus of claim 3, wherein the lower appetite control device is dimensioned to cover between eight and twelve lower teeth of the user.

6. The apparatus of claim 2, wherein the appetite control device is dimensioned to cover fourteen upper teeth of the user.

7. The apparatus of claim 2, wherein the appetite control device is dimensioned to cover between eight and twelve upper teeth of the user.

8. The apparatus of claim 3, further comprising:
a storage case, wherein the storage case is dimensioned to hold the appetite control device, the lower appetite control device, and one or more flavor tabs or flavor gels.

9. The apparatus of claim 2, wherein the appetite control device is made from a translucent plastic.

10. The apparatus of claim 2, wherein the appetite control device has a width of about 5.715 cm, a height of about 5.08 cm, and an inner width between the left end and the right ends of about 3.175 cm.

11. The apparatus of claim 2, wherein the appetite control device is wider proximate the left end and the right end.

12. The apparatus of claim 2, wherein the appetite control device comprises one or more apertures on an outer sidewall.

13. The apparatus of claim 2, wherein the appetite control device comprises one or more apertures on an inner sidewall.

14. The apparatus of claim 2, wherein the appetite control device comprises a generally trapezoidal cross section.

15. The apparatus of claim 2, wherein the appetite control device comprises a lower width of about 0.635 cm, an upper width of about 1.27 cm, and a height of about 0.47625 cm.

16. The apparatus of claim 2, wherein the flavor tab or flavor gel comprises nicotine.

17. The apparatus of claim 2, wherein a top surface of the flavor pouch is substantially level with an upper surface of the appetite control device.

18. The apparatus of claim 2, wherein a lower surface of the flavor pouch is substantially level with a lower surface of the appetite control device.

19. The apparatus of claim 2, wherein the flavor pouch is disposed between an upper surface and a lower surface of the appetite control device.

20. The apparatus of claim 2, wherein the appetite control device is made from a heat-cured acrylic resin.

21-41. (canceled)
